(19) United States
(12) Patent Application Publication
(10) Pub. No.: US 2020/0360317 A1
(43) Pub. Date: Nov. 19, 2020

(54) CARBOXYLIC ACIDS FOR TREATING/PREVENTING A SKIN DISEASE

(71) Applicant: Proponent Biotech GmbH, Zug (CH)

(72) Inventor: Bettina ERNST, Zurich (CH)

(73) Assignee: Proponent Biotech GmbH, Zug (CH)

(21) Appl. No.: 16/097,720

(22) PCT Filed: May 17, 2017
(86) PCT No.: PCT/EP2017/061826

§ 371 Date: Oct. 30, 2018

(30) Foreign Application Priority Data
May 17, 2016 (EP) 16169883.2

(51) Int. Cl. A61K 31/19; A61K 9/00; A61P 17/04; A61P 37/08; A61K 8/36

(52) U.S. Cl. CPC A61K 31/19; A61K 9/0014; A61K 9/0043; A61K 9/06; A61P 17/04; A61P 37/08; A61K 8/36; A61K 9/0053

(57) ABSTRACT

The present invention relates to a pharmaceutical composition comprising a carboxylic acid or a pharmaceutically acceptable salt thereof as active ingredient for use in treating and/or preventing a skin disease. The invention also relates to a method for treating and/or preventing a skin disease of a patient, the method comprising administering an effective amount of a carboxylic acid or a pharmaceutically acceptable salt thereof to a patient in need thereof. Furthermore, the invention provides cosmetic uses of a composition comprising a carboxylic acid or a salt thereof for reducing redness of the skin, skin lesions, wrinkles, impurities or irregularities of the skin. In addition, the present invention relates to a pharmaceutical composition comprising as active ingredient a carboxylic acid or a pharmaceutically acceptable salt thereof for use in improving barrier function of the skin, wherein the pharmaceutical composition is administered to an infant and a method for treating and/or preventing a skin disease of a patient, the method comprising administering an effective amount of a carboxylic acid or a pharmaceutically acceptable salt thereof to a patient in need thereof.

The present invention relates to a pharmaceutical composition comprising a carboxylic acid or a pharmaceutically acceptable salt thereof as active ingredient for use in treating and/or preventing a skin disease. The invention also relates to a method for treating and/or preventing a skin disease of a patient, the method comprising administering an effective amount of a carboxylic acid or a pharmaceutically acceptable salt thereof to a patient in need thereof. Furthermore, the invention provides cosmetic uses of a composition comprising a carboxylic acid or a salt thereof for reducing redness of the skin, skin lesions, wrinkles, impurities or irregularities of the skin. In addition, the present invention relates to a pharmaceutical composition comprising as active ingredient a carboxylic acid or a pharmaceutically acceptable salt thereof for use in improving barrier function of the skin, wherein the pharmaceutical composition is administered to an infant and a method for treating and/or preventing a skin disease of a patient, the method comprising administering an effective amount of a carboxylic acid or a pharmaceutically acceptable salt thereof to a patient in need thereof.

Skin diseases are among the most prevalent diseases in humans. Moreover, allergic reactions of the skin due to contact with an allergen are increasingly common among humans. Diseases such as atopic contact dermatitis are classically treated using methods comprising as a first step recognition of the clinical problem, followed by identification of the culprit chemical and the source of that chemical. In the meantime, the skin reaction can be temporized by cleansing the skin area in contact with the chemical, applying a damp cloth to cool the skin, and when necessary using topical corticosteroids to reduce the inflammation and antihistamines. Corticosteroid creams should be used carefully and according to the prescribed directions because when overused over longer periods of time they can cause thinning of the skin. Also, in some instances such as poison ivy dermatitis calamine lotion and cool oatmeal baths may relieve itching. Usually, severe cases are treated with systemic corticosteroids which may be tapered gradually, with various dosing schedules ranging from a total of 12-20 days to prevent the recurrence of the rash (while the chemical allergen is still in the skin, up to 3 weeks, as well as a topical corticosteroid. Tacrolimus ointment or pimecrolimus cream can also be used additionally to the corticosteroid creams or instead of these.

Oral antihistamines such as diphenhydramine or hydroxyzine may also be used in more severe cases to relieve the intense itching. Topical antihistamines are not advised as there might be a second skin reaction (treatment associated contact dermatitis) from the lotion itself.

Other symptoms caused by allergic contact dermatitis may be eased with cool compresses to stop the itching. It is vital for treatment success that the trigger be identified and avoided. The discomfort caused by the symptoms may be relieved by wearing smooth-textured cotton clothing to avoid frictional skin irritation or by avoiding soaps with perfumes and dyes.

Commonly, the symptoms may resolve without treatment in 2 to 4 weeks but specific medication may hasten the healing as long as the trigger is avoided.

In addition, it is known that the body's barrier surfaces, for example the skin, continue to develop in their ‘barrier function’ postnatally. As an example, the ability of fluid to traverse the skin reduces from birth over the first months/year of life as the skin barrier matures. This can be measured by Transepidermal Water Loss (TEWL), which progressively decreases following birth.

It is furthermore known that there are critical periods of time during development—the so-called “window of opportunity”—early in life, which are critical for the development of the immune system and, thus, general health of a subject during later life.

Thus, it is desirable to improve barrier function of the skin early in life in order to prevent serious diseases in early childhood. In addition, it is desirable to have a long-term effect of the improved barrier function during later life. Preferably, the barrier function should be improved by an agent, which can be administered conveniently and, thus, has good subject compliance.

In view of the foregoing and, in particular, the known drawbacks of corticosteroids, there is an urgent need for improved means and methods for treating and/or preventing skin diseases, in particular atopic contact dermatitis, that cause a quick reduction of symptoms.

Thus, the technical problem underlying the present invention is the provision of improved means and methods for treating/preventing a skin disease and/or reducing redness of the skin, skin lesions, wrinkles, impurities and/or irregularities of the skin. In addition, the present invention solves the technical problem of providing an agent, which improves barrier function in early childhood and has a long-term beneficial effect on barrier function of the skin.

The technical problem is solved by provision of the embodiments characterized in the claims.

Accordingly, the present invention relates to a carboxylic acid or a pharmaceutically acceptable salt thereof, particularly to a pharmaceutical composition comprising a carboxylic acid or a pharmaceutically acceptable salt thereof as active ingredient for use in treating and/or preventing a skin disease, particularly upon administration of said composition to a patient in need thereof, particularly wherein said patient is an animal or a human.

In various embodiments, the present invention relates to a carboxylic acid or a pharmaceutically acceptable salt thereof, particularly to a pharmaceutical composition comprising a carboxylic acid or a pharmaceutically acceptable salt thereof as active ingredient for use in treating a skin disease, particularly upon administration of said composition to a patient in need thereof, particularly wherein said patient is an animal or a human.

In various further embodiments, the present invention relates to a carboxylic acid or a pharmaceutically acceptable salt thereof, particularly to a pharmaceutical composition comprising a carboxylic acid or a pharmaceutically acceptable salt thereof as active ingredient for use in preventing a skin disease, particularly upon administration of said composition to a patient in need thereof, particularly wherein said patient is an animal or a human.

The present invention also relates to a carboxylic acid or a pharmaceutically acceptable salt thereof, particularly to a pharmaceutical composition comprising a carboxylic acid or a pharmaceutically acceptable salt thereof as active ingredient for use in alleviating the symptoms of the skin disease, particularly upon administration of said composition to a patient in need thereof, particularly wherein said patient is an animal or a human.

In a specific embodiment, administration of the carboxylic acid or a pharmaceutically acceptable salt thereof, particularly of a pharmaceutical composition comprising a carboxylic acid or a pharmaceutically acceptable salt thereof as active ingredient leads to a reduction of redness of the skin, skin lesions, wrinkles, impurities and/or irregularities of the skin.

In various embodiments of the invention, the pharmaceutical composition according to the invention and as described herein comprises a pharmaceutically acceptable carrier and/or excipient.

In various other embodiments of the invention, the carboxylic acid is the only active ingredient comprised in the pharmaceutical composition as described herein in the various embodiments or aspects.

In various further embodiments of the invention, the carboxylic acid comprises between two and four carbon atoms.

In a specific embodiment, the carboxylic acid is acetic acid, propionic acid or butyric acid, preferably propionic acid or a pharmaceutically acceptable salt thereof.

The pharmaceutical composition for use according to any one of the embodiments disclosed herein may be administered topically.

In various embodiments of the invention, the skin disease is skin disease is dermatitis or eczema, particularly an atopic dermatitis or a contact dermatitis, particularly an allergic contact dermatitis, particularly an allergic contact dermatitis due to metals, particulary chromium or nickel, preferably nickel.

In various embodiments, the invention provides a method for treating and/or preventing a skin disease of a patient and/or for alleviating the symptoms thereof, the method comprising administering an effective amount, particularly a therapeutically effective amount of a carboxylic acid or a pharmaceutically acceptable salt thereof, or a pharmaceutical composition comprising an effective amount, particularly a therapeutically effective amount of a carboxylic acid or a pharmaceutically acceptable salt thereof, to a patient in need thereof, particularly topically.

In particular, the invention provides a method for treating a skin disease of a patient, the method comprising administering an effective amount, particularly a therapeutically effective amount of a carboxylic acid or a pharmaceutically acceptable salt thereof, or a pharmaceutical composition comprising an effective amount, particularly a therapeutically effective amount of a carboxylic acid or a pharmaceutically acceptable salt thereof, to a patient in need thereof, particularly topically.

The invention further provides a method for preventing a skin disease of a patient, the method comprising administering an effective amount, particularly a therapeutically effective amount of a carboxylic acid or a pharmaceutically acceptable salt thereof, or a pharmaceutical composition comprising an effective amount, particularly a therapeutically effective amount of a carboxylic acid or a pharmaceutically acceptable salt thereof, to a patient in need thereof, particularly topically.

The invention also provides a method for alleviating the symptoms a skin disease of a patient, the method comprising administering an effective amount, particularly a therapeutically effective amount of a carboxylic acid or a pharmaceutically acceptable salt thereof, or a pharmaceutical composition comprising an effective amount, particularly a therapeutically effective amount of a carboxylic acid or a pharmaceutically acceptable salt thereof, to a patient in need thereof, particularly topically.

The patient to be treated may be an animal, particularly a human.

In various embodiments of the invention, the carboxylic acid or pharmaceutical composition is the carboxylic acid or composition as defined in any one of the embodiments or aspect described herein.

In various other embodiments of the invention, the skin disease is a disease as defined in any one of the embodiments or aspect described herein.

The present invention further provides a cosmetic use of a composition comprising a carboxylic acid or a salt thereof as described herein in the various embodiments for reducing redness of the skin, skin lesions, wrinkles, impurities and/or irregularities of the skin, particularly for improving softness and appearance of the skin.

In various embodiments of the invention, the carboxylic acid comprises between two and four carbon atoms.

In a specific embodiment, the carboxylic acid is acetic acid, propionic acid or butyric acid, preferably propionic acid or a salt thereof.

In addition, the present invention relates to a carboxylic acid or a pharmaceutically acceptable salt thereof, particularly to a pharmaceutical composition comprising as active ingredient a carboxylic acid or a pharmaceutically acceptable salt thereof, as described herein in the various embodiments, for use in improving barrier function of the skin, wherein the pharmaceutical composition is administered to an infant. The invention also relates to a method for treating and/or preventing a skin disease of a patient, the method comprising administering an effective amount of a carboxylic acid or a pharmaceutically acceptable salt thereof, particularly of a pharmaceutical composition comprising as active ingredient a carboxylic acid or a pharmaceutically acceptable salt thereof, as described herein in the various embodiments, to a patient in need thereof, particularly wherein said patient is an infant, particularly an infant of the age of 0 to 3 years.

In various embodiments of the invention, administration of the carboxylic acid or a pharmaceutically acceptable salt thereof, particularly of the pharmaceutical composition comprising as active ingredient a carboxylic acid or a pharmaceutically acceptable salt thereof, as described herein in the various embodiments, to an infant, protects the infant from infections and diseases transmitted via the skin.

In various embodiments of the invention, the carboxylic acid comprises between two and four carbon atoms, particularly the carboxylic acid is acetic acid, propionic acid or butyric acid, or a pharmaceutically acceptable salt thereof, preferably propionic acid, or a pharmaceutically acceptable salt thereof.

In various further embodiment of the invention, the carboxylic acid or a pharmaceutically acceptable salt thereof, particularly of the pharmaceutical composition comprising as active ingredient a carboxylic acid or a pharmaceutically acceptable salt thereof, as described herein in the various embodiments, is administered in liquid form, particularly in form of a spray, a gel, a cream or an aqueous solution, or solid form, particularly in form of a capsule or tablet.

In various further embodiments of the invention, the carboxylic acid or a pharmaceutically acceptable salt thereof, particularly of the pharmaceutical composition comprising as active ingredient a carboxylic acid or a pharmaceutically acceptable salt thereof, as described herein in the various embodiments, is administered topically or orally.

In a specific embodiment of the invention, the pharmaceutical composition for use according to any one of the various embodiments described herein comprises the carboxylic acid as the only active ingredient.

In various embodiments, the invention provides a method for preventing a skin disease of a patient, the method comprising administering an effective amount of a carboxylic acid or a pharmaceutically acceptable salt thereof, or of a pharmaceutical composition comprising an effective amount of a carboxylic acid or a pharmaceutically acceptable salt thereof, to a patient, wherein the patient is an infant.

In another specific embodiment of the invention, the carboxylic acid or a pharmaceutically acceptable salt thereof, particularly the pharmaceutical composition comprising as active ingredient the carboxylic acid or a pharmaceutically acceptable salt thereof, is applied in liquid or solid form as defined herein in the various embodiments.

In still another specific embodiment of the invention, the carboxylic acid or a pharmaceutically acceptable salt thereof, particularly the pharmaceutical composition comprising as active ingredient the carboxylic acid or a pharmaceutically acceptable salt thereof is administered topically, intranasally or orally.

In a specific embodiment, the carboxylic acid is the carboxylic acid as defined herein in the various embodiments and the pharmaceutical composition is the composition as defined in the various embodiments.

Thus, it was surprisingly and unexpectedly found by the present inventors that a carboxylic acid may be used for the treatment of the skin, in particular by topical application, in particular by using a solution containing propionate. In this regard, human volunteers were topically administered a propionate solution subsequent to exposure to Nickel; see Example 1. Symptoms of dermatitis and/or eczema were observed. However, symptoms were significantly reduced, i.e. the clinical score was significantly reduced, subsequent to administration of a carboxylic acid, in particular propionate. Accordingly, the compositions and methods of the invention as described herein in the various embodiments or aspects are, inter alia, effective following contact hypersensitivity to nickel. In addition, compositions and methods of the invention as described herein in the various embodiments or aspects were effective in improving skin repair following an abrasion. Overall, treatment of skin with a solution containing propionate is effective at dampening skin diseases such as dermatitis and/or eczema and reducing redness of the skin, skin lesions, wrinkles, impurities and/or irregularities of the skin. The composition of the invention as described herein in the various embodiments or aspects can, inter alia, be applied topically and is relevant for the healthcare and cosmetics industry.

In addition, it has been surprisingly and unexpectedly been found by the present inventors that a carboxylic acid has beneficial effects on the barrier function of the skin and can safely be applied to infants. Moreover, it has surprisingly and unexpectedly been found that continuous administration of carboxylic acid starting in early childhood can have long-term beneficial effects by improving the barrier function throughout life.

Thus, in addition to the immediate effect of a carboxylic acid upon the improvement of barrier function, if the carboxylic acid is administered to barrier tissue, such as the skin, early in life, they can improve barrier function and consequently protect individuals against infections and disease, for prolonged periods throughout life.

This scientific discovery includes the exposure to a carboxylic acid topically early in life as a means of improving barrier function and consequently protection against infections, chemical-induced pathology and allergy.

In this regard, experimental evidence shows that treatments early in life (first days to 3 years) can have a profound beneficial impact on disease development throughout life. In addition, carboxylic acids can influence epithelial cells function and associated stromal and immune cells to effect tissue barrier function and protection against infection, chemical and allergen exposures.

The carboxylic acid used in the present invention is not particularly limited as long as it is an organic compound that contains a carboxyl group (C(O)OH) and having the general formula of R—C(O)OH, wherein R is a rest attached to the C(O)OH functional group. In particular embodiments of the present invention, R is an alkyl group, optionally having further modifications. In more particular embodiments, R represents a methyl, ethyl or propyl side chain. In a particular embodiment of the present invention, R is an ethyl side chain, the carboxylic acid thus being propionic acid.

Within the meaning of the present invention, the term “alkyl” as such means a straight-chained or branched saturated aliphatic hydrocarbon having from 1 to 10, in particular 1 to 3, carbon atoms, wherein the alkyl group may be unsubstituted or substituted with one or more, same or different, substituents selected from the group consisting of hydroxyl, amino, carboxylic acid, halogen, cyano, or nitro. Preferred are C₁-C₆ alkyl, such as methyl, ethyl, n-propyl, isopropyl, n-butyl, isobutyl, tert-butyl, n-pentyl (amyl), 2-pentyl (sec-pentyl), 3-pentyl, 2-methylbutyl, 3-methylbutyl (=iso-pentyl or iso-amyl), 3-methylbut-2-yl, 2-methylbut-2-yl, 2,2-dimethylpropyl (=neopentyl), n-hexyl, iso-hexyl, sec.-hexyl, tert.-hexyl and the like. Most preferred are C₁-C₃ alkyl, such as methyl, ethyl, n-propyl, isopropyl.

In accordance with the above, in still further specific embodiments, the carboxylic acid according to the invention and as described herein in the various embodiments is selected from the group consisting of acetic acid, propionic acid, butyric acid, isobutyric acid, 2-hydroxyproirinic acid, dilactic acid, 2-benzyloxypropionic acid, 2-(p-nitrophenyl)-oxy-propionic acid, 3-hydroxypropionic acid, 2,3-dihydroxypropionic acid, methyl 3-hydroxypropionate, ethyl 3-hydroxypropionate, propyl 3-hydroxypropionate, benzyl 3-hydroxypropionate, para-nitrophenyl 3-hydroxypropionate, p-nitrobenzyl 3-hydroxypropionate, polyethylene glycol 3-hydroxypropionate, methyl propionate, ethyl propionate, propyl propionate, benzyl propionate, p-nitrophenyl propionate, p-nitrobenzyl propionate, 2-(4-Isobutylphenyl) propionic acid; or pharmaceutically acceptable salts thereof.

In a particular embodiment of the invention, the compound for use according to the invention and as described herein in the various embodiments is selected from the group consisting of acetic acid, propionic acid and butyric acid, or pharmaceutically acceptable salts thereof.

The carboxylic acid used in the present invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e. g., carbon-carbon bonds) wherein bond rotation is restricted about that particular linkage, e. g. restriction resulting from the presence of a ring or double bond. Accordingly, all cis-trans and E/Z isomers are expressly included in the present invention. The compounds of this invention may also be represented in multiple tautomeric forms, in such instances, the invention expressly includes all tautomeric forms of the compounds described herein, even though only a single tautomeric form may be represented (e. g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The carboxylic acid used in the present invention, or the pharmaceutically acceptable salt thereof, or a composition comprising the carboxylic acid according to the invention and as described herein in the various embodiments, particularly in a therapeutically effective amount, optionally, together with a pharmaceutically acceptable carrier, is used in the treatment and/or prevention of a skin disease.

Accordingly, in one embodiment, the present invention relates to a carboxylic acid, in particular acetic acid, propionic acid or butyric acid, according to the invention and as described herein in the various embodiments or to a pharmaceutically acceptable salt thereof, or to a composition comprising the carboxylic acid according to the invention and as described herein in the various embodiments, or a pharmaceutically acceptable salt thereof, particularly in a therapeutically effective amount, optionally, together with a pharmaceutically acceptable carrier, for use in the treatment and/or prevention of a skin disease.

In a yet further embodiment, the present invention relates to a carboxylic acid, in particular acetic acid, propionic acid or butyric acid, according to the invention and as described herein in the various embodiments or to a pharmaceutically acceptable salt thereof, or to a composition comprising the carboxylic acid according to the invention and as described herein in the various embodiments, or a pharmaceutically acceptable salt thereof, particularly in a therapeutically effective amount, optionally, together with a pharmaceutically acceptable carrier, for use in improving the barrier function of the skin, particularly of an infant.

There are various diseases known, which are associated with a reduced barrier function of the skin. However, diseases to be treated/prevented by the means provided in the present invention are not limited to those diseases directly associated with a reduced barrier function of the skin, but also extend to diseases, which are a consequence of a reduced barrier function, as e.g. diseases caused by agents exhibiting an effect on the skin due to a reduced barrier function thereof, e.g. allergens or toxic agents.

Thus, skin diseases within the meaning of the present invention are those internationally classified in ICD-10 chapter XII comprising those in blocks L00 to L99. In particular, skin diseases treated and/or prevented by the means of the present invention are those of blocks L20 to L30, i.e. dermatitis and eczema. Thus, diseases treated and/or prevented by the means of the present invention comprise atopic dermatitis, comprising Besnier's prurigo; seborrhoeic dermatitis comprising seborrhoea capitis, cradle cap; diaper (napkin) dermatitis comprising diaper rash; allergic contact dermatitis comprising allergic contact dermatitis due to metals, allergic contact dermatitis due to adhesives, allergic contact dermatitis due to cosmetics, allergic contact dermatitis due to drugs in contact with skin, allergic contact dermatitis due to dyes, allergic contact dermatitis due to other chemical products, allergic contact dermatitis due to food in contact with skin, allergic contact dermatitis due to plants, except food, allergic contact dermatitis due to other agents; irritant contact dermatitis comprising irritant contact dermatitis due to detergents, irritant contact dermatitis due to oils and greases, irritant contact dermatitis due to solvents, irritant contact dermatitis due to cosmetics, irritant contact dermatitis due to drugs in contact with skin, irritant contact dermatitis due to other chemical products, irritant contact dermatitis due to food in contact with skin, irritant contact dermatitis due to plants, except food, irritant contact dermatitis due to other agents; unspecified contact dermatitis comprising unspecified contact dermatitis due to cosmetics, unspecified contact dermatitis due to drugs in contact with skin, unspecified contact dermatitis due to dyes, unspecified contact dermatitis due to other chemical products, unspecified contact dermatitis due to food in contact with skin, unspecified contact dermatitis due to plants, except food, unspecified contact dermatitis due to other agents; exfoliative dermatitis; dermatitis due to substances taken internally comprising generalized skin eruption due to drugs and medicaments, localized skin eruption due to drugs and medicaments, dermatitis due to ingested food, dermatitis due to other substances taken internally, dermatitis due to unspecified substance taken internally; Lichen simplex chronicus and prurigo comprising Lichen simplex chronicus, prurigo nodularis, other prurigo; pruritus comprising pruritus ani, pruritus scroti, pruritus vulvae, anogenital pruritus, other pruritus; other dermatitis comprising nummular dermatitis, Dyshidrosis (pompholyx), cutaneous autosensitization, candidid, dermatophytid, eczematid, infective dermatitis, erythema intertrigo, pityriasis alba, other specified dermatitis or eczema.

In a specific embodiment of the present invention, the disease is an atopic dermatitis or a contact dermatitis, in particular an allergic contact dermatitis, more particularly an allergic contact dermatitis due to metals.

The term “allergic contact dermatitis” (ACD) as used herein refers to a form of contact dermatitis that is the manifestation of an allergic response caused by contact with a substance.

ACD is accepted to be the most prevalent form of immunotoxicity found in humans. ACD is a hypersensitive reaction that is atypical within the population.

The symptoms of allergic contact dermatitis are very similar to the ones caused by irritant contact dermatitis. The first sign of allergic contact dermatitis is the presence of the rash or skin lesion at the site of exposure. Depending on the type of allergen causing it, the rash can ooze, drain or crust and it can become raw, scaled or thickened. It is possible that the skin lesion does not take the form of a rash but it may include papules, blisters, vesicles or even a simple red area. The main difference between the rash caused by allergic contact dermatitis and the one caused by irritant contact dermatitis is that the first one tends to be confined to the area where the trigger touched the skin, whereas in the second case, the rash is more likely to be more widespread on the skin. Sometimes, allergic contact dermatitis rash only appears after a day or two after exposure to the allergen.

Other symptoms may include itching, skin redness or inflammation, localized swelling and the area may become more tender or warmer. If left untreated, the skin may darken and become leathery and cracked. Pain can also be present.

The symptoms of allergic contact may persist for as long as one month before resolving completely. Once an individual has developed a skin reaction to a certain substance it is most likely that they will have it for the rest of their life, and the symptoms will reappear when in contact with the allergen.

Allergic contact dermatitis may be caused by a variety of agents including but not limited to Nickel (nickel sulfate hexahydrate), which is the most frequently confirmed contact allergen worldwide. Nickel is frequently encountered in jewelry and clasps or buttons on clothing. Allergy to nickel cost more than a billion dollars globally each year. A further cause may be gold (gold sodium thiosulfate), which is also often found in jewelry and dental materials, Chromium is also a frequent cause of allergic contact dermatitis. Chromium is used in the tanning of leather. Also a component of uncured cement/mortar, facial cosmetics and some bar soaps. Other agents include the oily coating from plants of Toxicodendron genus, sap from certain species of mangrove and agave, Thiomersal, Neomycin, fragrances, formaldehyde, cobalt chloride, bacitracin, quaternium-15, photographic developers, especially those containing metol, topical anesthetics such as pramoxine or diphenhydramine, after prolonged use, isothiazolinones, mercaptobenzothiazole, or soluble salts of platinum. Within the present invention, the ACD to be treated and/or prevented preferably is one caused by contact with a metal, in particular Nickel.

Diagnosing allergic contact dermatitis is primarily based on physical exam and medical history. However, the present invention is not limited to treating and/or preventing ACDs diagnosed by such methods. In some cases doctors can establish an accurate diagnosis based on the symptoms that the patient experiences and on the rash's appearance. In the case of a single episode of allergic contact dermatitis, this is all that is necessary. Chronic and/or intermittent rashes which are not readily explained by history and physical exam often will benefit from further testing.

Hypersensitivity allergy test is a commonly used examination to determine the exact cause of an allergic contact dermatitis. According to the American Academy of Allergy, Asthma, and Immunology, “patch testing is the gold standard for contact allergen identification”.

The patch test consists of applying small quantities of potential allergens to small patches and which are then placed on the skin. After two days, they are removed and if a skin reaction occurred to one of the substances applied, a raised bump will be noticeable underneath the patch. The tests are again read at 72 or 96 hours after application.

Patch testing is used for patients who have chronic, recurring contact dermatitis. Other tests that may be used to diagnose contact dermatitis and rule out other potential causes of the symptoms include a skin biopsy and culture of the skin lesion.

Thus, in one specific embodiment, the present invention relates to a pharmaceutical composition comprising a carboxylic acid, in particular acetic acid, propionic acid or butyric acid, according to the invention and as described herein in the various embodiments or to a pharmaceutically acceptable salt thereof, or to a carboxylic acid according to the invention and as described herein in the various embodiments, or a pharmaceutically acceptable salt thereof, particularly in a therapeutically effective amount, optionally, together with a pharmaceutically acceptable carrier, for use in the treatment and/or prevention of an allergic contact dermatitis due to metals, in particular wherein the metal is chromium or nickel, more particularly nickel.

In a further specific embodiment, the present invention relates to a pharmaceutical composition comprising a carboxylic acid, in particular acetic acid, propionic acid or butyric acid, according to the invention and as described herein in the various embodiments or to a pharmaceutically acceptable salt thereof, or to a carboxylic acid according to the invention and as described herein in the various embodiments, or a pharmaceutically acceptable salt thereof, particularly in a therapeutically effective amount, optionally, together with a pharmaceutically acceptable carrier, for use in improving barrier function of the skin of an infant and thereby treating and/or preventing an allergic contact dermatitis due to metals, in particular wherein the metal is chromium or nickel, more particularly nickel, wherein the pharmaceutical composition comprising a carboxylic acid or a pharmaceutically acceptable salt thereof is administered to an infant, preferably wherein the pharmaceutical composition is administered to the infant regularly starting from its birth.

Pharmaceutical acceptable carriers are well-known in the art. That is, the person skilled in the art can easily obtain an acceptable carrier for use with the means and methods of the present invention. The pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, and polyvinyl pyrrolidone. The carrier may also comprise any of the substances described in Remington: The Science and Practice of Pharmacy (Gennaro and Gennaro, Eds, 20th edition, Lippincott Williams & Wilkins, 2000); Theory and Practice of Industrial Pharmacy (Lachman et al, eds., 3^{rd} edition, Lippincott Williams & Wilkins, 1986); Encyclopedia of Pharmaceutical Technology (Swarbrick and Boylan, eds., 2nd edition, Marcel Dekker, 2002). The fillers can be chosen from, but are not limited to, powdered cellulose, sorbitol, mannitol, various types of lactose, phosphates and the like.

The polymers can be chosen from, but not limited to, hydrophilic or hydrophobic polymers such as derivatives of cellulose (for example methylcellulose, hydroxypropyl cellulose, hypromellose, ethylcellulose); polyvinylpirolidone (for example povidone, crospovidone, copovidone); polymethacrylates (for example Eudragit RS, RL); lypophillic components (for example glyceryl monostearate, glyceryl behenate); and various other substances such as for example hydroxypropyl starch, polyethylene oxide, carrageenan and the like. Most commonly, hydrophilic swelling polymers of suitable viscosity such as hypromellose are used, preferably in amounts above 5%, and more preferably above 8%. Glidants can be chosen from, but not limited to, colloidal silicon dioxide, talc, magnesium stearate, calcium stearate, aluminium stearate, palmitic acid, stearic acid, stearol, cetanol, polyethylene glycol and the like. Lubricants can be chosen from, but not limited to, stearic acid, magnesium stearate, calcium stearate, aluminium stearate, sodium stearyl fumarate, talc, hydrogenated castor oil, polyethylene glycols and the like.

The active ingredients of the present invention can be formulated into the composition as neutralized pharmaceutically acceptable salt forms. Pharmaceutically acceptable salts include, but are not limited to, the acid addition salts, which are formed with inorganic acids such as, for example, hydrochloric, sulfuric or phosphoric acids, or such organic acids as acetic, oxalic, tartaric, mandelic, citric and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In a specific embodiment of the invention, the pharmaceutical composition is for topical administration, i.e. it is a topical composition. Topical compositions useful in the present invention involve formulations suitable for topical application to skin. In one embodiment, the composition comprises the carboxylic acid or the salt thereof and a pharmaceutically-acceptable topical carrier. In one embodiment, the pharmaceutically-acceptable topical carrier is from about 50%© to about 99.99%, by weight, of the composition (e.g., from about 80% to about 95%, by weight, of the composition.

The compositions may be made into a wide variety of product types that include but are not limited to lotions, creams, gels, sticks, sprays, shaving creams, ointments, cleansing liquid washes and solid bars, shampoos, pastes, powders, mousses, shaving creams, wipes, patches, nail lacquers, wound dressing, adhesive bandages, hydrogels, films and make-up such as concealers, foundations, mascaras, and lipsticks. These product types may comprise several types of pharmaceutically-acceptable topical carriers including, but not limited to solutions, emulsions (e.g., microemulsions and nanoemulsions), gels, solids, micelles, and liposomes. The following are non-limitative examples of such topical carriers. Other topical carriers can be formulated by those of ordinary skill in the art.

The topical compositions useful in the present invention can be formulated as solutions. Solutions typically include an aqueous solvent (e.g., from about 50% to about 99.99%, such as from about 90%© to about 99%©, by weight of a pharmaceutically acceptable aqueous solvent). Topical compositions useful in the subject invention may be formulated as a solution comprising an emollient. Such compositions preferably contain from about 2% to about 50%© of an emollient (s). As used herein, “emollients” refer to materials used for the prevention or relief of dryness, as well as for the protection of the skin. A wide variety of suitable emollients are known and may be used herein. See the International Cosmetic Ingredient Dictionary and Handbook, eds. Wenninger and McEwen, pp. 1656-61, 1626, and 1654-55 (The Cosmetic, Toiletry, and Fragrance Assoc, Washington, D.C., 7^{th} Edition, 1997) (hereinafter “ICI Handbook”) contains numerous examples of suitable materials.

A lotion can be made from such a solution. Lotions typically comprise from about 1% to about 20% (e.g., from about 5% to about 10%©) of an emollient (s) and from about 50% to about 90% (e.g., from about 60% to about 80%) of water.

Another type of product that may be formulated from a solution is a cream. A cream typically comprises from about 5% to about 50% (e.g., from about 10% to about 20%©) of an emollient (s) and from about 45% to about 85% (e.g., from about 50% to about 75%) of water.

Yet another type of product that may be formulated from a solution is an ointment. An ointment may comprise a simple base of animal or vegetable oils or semi-solid hydrocarbons. An ointment may comprise from about 2% to about 10% of an emollient (s) plus from about 0.1% to about 2% of a thickening agent (s). A more complete disclosure of thickening agents or viscosity increasing agents useful herein can be found in the ICI Handbook pp. 1693-1697. The topical compositions useful in the present invention can also be formulated as emulsions. If the carrier is an emulsion, from about 1% to about 10% (e.g., from about 2% to about 5%) of the carrier comprises an emulsifier (s). Emulsifiers may be nonionic, anionic or cationic. Suitable emulsifiers are disclosed in the ICI Handbook, pp 0.1673-1686.

Lotions and creams can be formulated as emulsions. Typically, such lotions comprise from 0.5% to about 5% of an emulsifier (s). Such creams would typically comprise from about 1% to about 20% (e.g., from about 5% to about 10%) of an emollient (s); from about 20% to about 80% (e.g., from 30% to about 70%) of water; and from about 1% to about 10% (e.g., from about 2% to about 5%) of an emulsifier (s) Single emulsion skin care preparations, such as lotions and creams, of the oil-in-water type and water-in-oil type are well-known in the cosmetic art and are useful in the subject invention. Multiphase emulsion compositions, such as the water-in-oil-in-water type are also useful in the subject invention. In general, such single or multiphase emulsions contain water, emollients, and emulsifiers as essential ingredients.

The topical compositions of this invention can also be formulated as a gel (e.g., an aqueous gel using a suitable gelling agent (s). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprises between about 0.1% and 5%, by weight, of such gelling agents.

The topical compositions of the present invention can also be formulated into a solid formulation (e.g., a wax-based stick, soap bar composition, powder, or a wipe containing powder).

Liposomal formulations are also useful compositions of the subject invention. Examples of liposomes are unilamellar, multilamellar, and paucilamellar liposomes, which may or may not contain phospholipids. Liposomes typically have size from about 50 nm to about 10 microns, such as about 0.1 to about 1 microns. Such compositions can be prepared by first combining the carboxylic acid with a phospholipid, such as dipalmitoylphosphatidyl choline, cholesterol and water. Epidermal lipids of suitable composition for forming liposomes may be substituted for the phospholipid. Examples of such epidermal lipids include, but are not limited to, glyceryl monoesters and diesters, polyethylene fatty ethers, and sterols. The liposome preparation may then incorporated into one of the above carriers (e.g., suspended in a solution, gel, or an oil-in-water emulsion) in order to produce the liposomal formulation.

Micelle formulations are also useful compositions of the subject invention. Such compositions can be prepared using single chain surfactants and lipids.

Micelles typically have size from about 1 nm to about 100 nm, such as from about 10 nm to about 50 nm. The micelle preparation may then incorporated into one of the above carriers (e.g., a gel or a solution) in order to produce the micelle formulation.

The topical compositions useful in the subject invention may contain, in addition to the aforementioned components, a wide variety of additional oil-soluble materials and/or water-soluble materials conventionally used in compositions for use on skin, hair, and nails at their art-established levels.

Irrespective of the pharmaceutically acceptable carrier used in the present invention or the formulation of the pharmaceutical composition of the invention, it is preferred that the carboxylic acid or the acceptable salt thereof is the only active ingredient comprised in said pharmaceutical composition. The term “active ingredient” within the meaning of the invention, is a pharmaceutical active substance, in particular the carboxylic acid, i.e. a substance that shows a physiological effect when it is absorbed in sufficient amount by the body of an organism. The physiological effect within the meaning of the invention is a reduction of clinical score of the skin disease, in particular the atopic dermatitis or eczema, in particular as determined using the methods shown in the appended Examples or using methods known to those skilled in the art.

In a further aspect, the present invention relates to a method for treating and/or preventing a skin disease of a patient, the method comprising administering an effective amount of a carboxylic acid according to the invention and as described herein in the various embodiments, or a pharmaceutically acceptable salt thereof, to a patient in need thereof.

The present invention also relates to a method for alleviating the symptoms of a skin disease of a patient, the method comprising administering an effective amount of a carboxylic acid according to the invention and as described herein in the various embodiments, or a pharmaceutically acceptable salt thereof, to a patient in need thereof.

An effective amount refers to that amount which provides a therapeutic effect for a given condition and administration regimen. In particular, “therapeutically effective amount” means an amount that is effective to prevent, alleviate or ameliorate symptoms of the disease or, in particular, reduce the clinical score of the skin disease in a human or non-human animal. Determination of a therapeutically effective amount is within the skill of the person skilled in the art. The therapeutically effective amount or dosage of a compound according to this invention can vary within wide limits and may be determined in a manner known in the relevant art. The dosage can vary within wide limits and will, of course, have to be adjusted to the individual requirements in each particular case.

In a further aspect, the present invention relates to a method for improving the barrier function of the skin of a subject, in particular an infant, the method comprising administering an effective amount of a carboxylic acid according to the invention and as described herein in the various embodiments, or a pharmaceutically acceptable salt thereof to a subject. An effective amount refers to that amount which provides a therapeutic effect for a given condition and administration regimen. In particular, “therapeutically effective amount” means an amount that is effective to prevent, alleviate or ameliorate symptoms of the disease or, in particular, reduce the clinical score of the skin disease in a human or non-human animal. Determination of a therapeutically effective amount is within the skill of the person skilled in the art. The therapeutically effective amount or dosage of a compound according to this invention can vary within wide limits and may be determined in a manner known in the relevant art. The dosage can vary within wide limits and will, of course, have to be adjusted to the individual requirements in each particular case.

In this regard, the terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease related to an undesired immune response from occurring in a subject which may be predisposed to the disease; (b) inhibiting the disease, i.e. arresting its development; or (c) relieving the disease, i.e. causing regression of the disease.

A “patient” or “subject” for the purposes of the present invention is used interchangeably and meant to include both humans and other animals, particularly mammals, and other organisms. Thus, the methods are applicable to both human therapy and veterinary applications. In the preferred embodiment the patient or subject is a mammal, and in the most preferred embodiment the patient or subject is a human.

The term “propionate” refers to the pharmaceutically acceptable salt of propionic acid such as, for example, the sodium salt of propionic acid.

The term “pharmaceutically acceptable salts” include salts of acidic or basic groups present in compounds of the invention as described herein in the various embodiments or aspects. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts.

The expressions “pharmaceutical composition” and “therapeutical composition” are used herein interchangeably in the widest sense. They are meant to refer, for the purposes of the present invention, to a therapeutically effective amount of the active ingredient, i.e. the carboxylic acid or a pharmaceutically acceptable salt thereof, optionally, together with a pharmaceutically acceptable carrier or diluent.

It embraces compositions that are suitable for the curative treatment, the control, the amelioration, an improvement of the condition or the prevention of a disease or disorder in a human being or a non-human animal. Thus, it embraces pharmaceutical compositions for the use in the area of human or veterinary medicine. Such a “therapeutic composition” is characterized in that it embraces a carboxylic acid or a physiologically acceptable salt thereof, and optionally a carrier or excipient whereby the salt and the carrier and excipient are tolerated by the target organism that is treated therewith.

The compounds of the present invention and as described herein in the various embodiments and the pharmaceutical compositions containing said compounds may be administered topically to body surfaces and thus be formulated in a form suitable for topical administration, as described above.

The pharmaceutical compositions provided herein in the various embodiments may also be administered as controlled-release compositions, i.e. compositions in which the active ingredient is released over a period of time after administration. Controlled- or sustained-release compositions include formulation in lipophilic depots (e.g. fatty acids, waxes, oils). In another embodiment, the composition is an immediate-release composition, i.e. a composition in which all the active ingredient is released immediately after administration.

The pharmaceutical compositions as described herein in the various embodiments may be used in human and veterinary medicine for treating humans and animals, including avians, non-human primates, dogs, cats, pigs, goats, sheep, cattle, horses, mice, rats and rabbits. It is preferred to treat humans.

Suitable dosages of the pharmaceutical compositions according to the invention and as described herein in the various embodiments will vary depending upon the condition, age and species of the subject, and can be readily determined by those skilled in the art. Such dosage will be adjusted to the individual requirements in each particular case including the specific compound(s) being administered, the route of administration, the condition being treated, as well as the patient being treated. However, the compounds can also be administered as depot preparations (implants, slow-release formulations, etc.) weekly, monthly or at even longer intervals. A particular preparation is a plaster, patch or the like. In such cases the dosage will be much higher than the daily one and has to be adapted to the administration form, the body weight and the concrete indication. The appropriate dosage can be determined by conducting conventional model tests, preferably animal models. The daily dosage can be administered as a single dose or in divided doses.

An effective dose of active ingredient(s) depends at least on the nature of the condition being treated, toxicity, whether the compound(s) is being used prophylactically (lower doses) or against an active condition, the method of delivery, and the pharmaceutical formulation, and will be determined by the clinician using conventional dose escalation studies.

In a particular embodiment, the pharmaceutical composition of the invention as described herein in the various embodiments or aspects is administered daily over an extended period of time to an infant. Regular application, in particular daily application, has a beneficial long-term effect of preventing diseases from developing due to an improved barrier function of the skin. Thus, a regular, in particular daily, application can prevent that agents can pass through the skin into the subject's body due to an improved barrier function of the skin caused by the application of the pharmaceutical composition of the invention. Such a long-term beneficial effect is observed for infants during their entire life-span. To maximize such effects, it is preferred to start administering a daily dose of the pharmaceutical composition of the invention as described herein in the various embodiments or aspects early in life, i.e. preferably immediately after birth, at the age of 1, 2, 3, 4, 5, 6, 7, 8 weeks or 2, 3, 4, 5, 6, 7, 8, 9, 10, 11, 12 months after birth.

In a further aspect of the present invention, the cosmetic use of a composition comprising a carboxylic acid according to the invention and as described herein in the various embodiments or a salt thereof for reducing redness of the skin, skin lesions, wrinkles, impurities and/or irregularities of the skin is provided. As such, the carboxylic acid is used as cosmetically active agent as part of a cosmetic composition. In particular, the carboxylic acid may be the only cosmetically active agent as part of the cosmetic composition.

The term “cosmetically active agent”, as used herein, refers to a compound (e.g., a synthetic compound or a compound isolated from a natural source or a natural extract), in particular the carboxylic acid, in particular acetic acid, propionic acid or butyric acid, that has a cosmetic or therapeutic effect on the skin or hair, including, but not limiting to, anti-acne agents, shine control agents, anti-mycotic agents, anti-parasite agents, external analgesics, sunscreens, photoprotectors, antioxidants, keratolytic agents, surfactants, moisturizers, vitamins, energy enhancers, anti-perspiration agents, astringents, deodorants, anti-callous agents, and agents for hair and/or skin conditioning. In particular, the cosmetically active agent, i.e. the carboxylic acid, will reduce redness of the skin, skin lesions, wrinkles, impurities and/or irregularities of the skin. The term “cosmetic composition” as used herein refers in particular to cosmetic compositions that can be topically applied to mammalian keratinous tissue such as e.g. human skin or scalp. The term “cosmetic composition” as used in the present application refers to cosmetic compositions as defined under the heading “Kosmetika” in Rompp Lexikon Chemie, 10th edition 1997, Georg Thieme Verlag Stuttgart, New York as well as to cosmetic compositions as disclosed in A. Domsch, “Cosmetic Compositions”, Verlag fur chemische Industrie (ed. H. Ziolkowsky), 4th edition, 1992.

As used herein, the term “cosmetically acceptable” modifying a substance means that the substance is of sufficiently high purity and suitable for use in contact with human skin without undue toxicity, incompatibility and instability. A “cosmetically acceptable” substance, preferably, causes little or no allergic response.

Also provided are skin care agents comprising the carboxylic acid according to the invention and as described herein in the various embodiments and as used herein. The term “skin care agent” refers to an agent that has one or more beneficial effects on the care and/or hygiene of the skin. It is to be understood that skin care active agents are useful not only for application to skin, but also to hair, scalp, nails and other mammalian keratinous tissue.

“Skin care composition”, as used herein, refers to a cosmetic composition comprising one or more skin care agents. The cosmetic composition as used herein may also cause a smoothing and softening of the skin due to reduction of symptoms as described above.

The terms “smoothing” and “softening” as used herein mean altering the surface of the skin and/or keratinous tissue such that its tactile feel is improved.

As used herein, the term “wound” relates to a body lesion with a discontinuity of the normal integrity of the tissue structures. That is, the cosmetic composition of the present invention may be used for reducing appearances of wounds.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While aspects of the invention are illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below. The invention also covers all further features shown in the figures individually, although they may not have been described in the previous or following description. Also, single alternatives of the embodiments described in the figures and the description and single alternatives of features thereof can be disclaimed from the subject matter of the other aspect of the invention.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

The patent or application file contains at least one drawing executed in color. Copies of this patent or patent, application publication with color drawing(s) will be provided by the Office upon request and payment of the necessary fee.

The present invention is also illustrated in some aspects by the following figures.

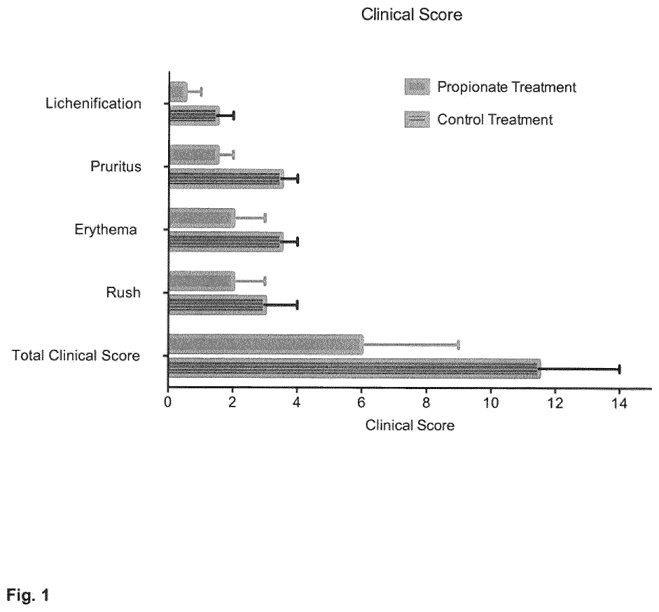

FIG. 1—Clinical score of different symptoms of contact dermatitis. The clinical score was determined following contact of the skin with Nickel. The figure shows a comparison of clinical score between skin treated with propionate and control treatment.

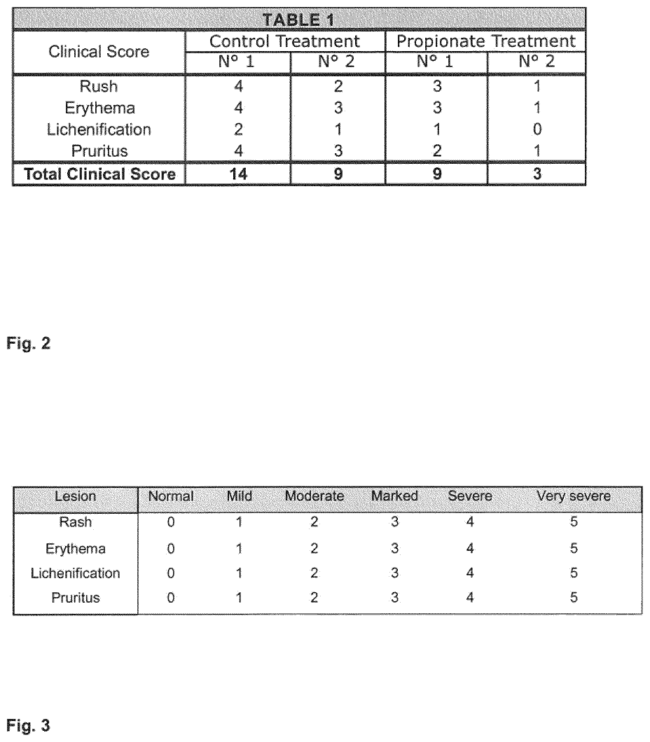

FIG. 2—Clinical score values. The table shows clinical score values of patients receiving propionate treatment or control treatment following Nickel exposure of the skin.

FIG. 3—Clinical score scheme. The table shows clinical score values for different severity levels of symptoms of skin lesions following Nickel exposure.

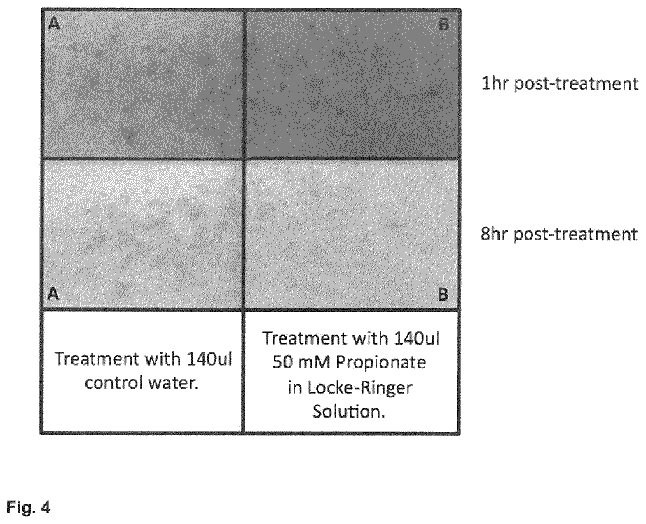

FIG. 4—Phonotypical appearance of skin lesions. The image illustrates differences between skin treated with water and skin treated with propionate. As can clearly be seen, lesions are significantly reduced when applying propionate to the skin.

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention. A number of documents including patent applications, manufacturer's manuals and scientific publications are cited herein. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### EXAMPLE 1—CARBOXYLIC ACID IN THE TREATMENT OF THE SKIN

The study aimed at determining whether treatment with propionate promotes skin health following allergic hypersensitivity (particularly allergic contact dermatitis).

Allergic hypersensitivity was induced through contact sensitization with Nickel allergen over a 12 hour time period. 60 hours following last contact with allergen, treatment of inflamed skin occurred over 8 hours with either 4×140 μl of 50 mM Propionate in Locke-Ringer solution or with 4×140 μl Locke-Ringer control solution.

### Preparation of Solution

After weighing of the appropriate amount of each chemical substance utilizing an analytical balance (Mettler Toledo GmbH, 8606 Greifensee, Switzerland), the chemical substances were added into a glass beaker. Thereafter H2O was added to requested final volume and the solution was mixed utilizing a magnetic stirrer. Immediately after the mixing of the solution, sterile filtration occurred utilizing a 0.22 μm filter (Stericup-GP, 0.22 μm, Polyethersulfon, 150 ml, gamma-sterilized, Catalogue number SCGPU01 RE, Merck Millipore Corporation, Merck KGaA, Darmstadt, Germany).

### The Locke-Ringer Solution had the Following Composition

TABLE 1

| Ingredient | mg/ml | Company | Catalogue N° | Lot N° |
|---|---|---|---|---|
| NaCl | 9 mg | Sigma-Aldrich | S5886-500G | SZBE3170 |
| KCl | 0.42 mg | Sigma-Aldrich | P5405-500G | SL8H5524V |
| CaCl₂ 2H₂0 | 0.32 mg | Sigma-Aldrich | C8106-100G | SLBH5904V |
| Dextrose | 2 mg | Sigma-Aldrich | D9434-250G | SLBH3471V |
| NaHCO₃ | 0.2 mg | Sigma-Aldrich | S5761-500G | SLBM8267V |
| Sterile H₂0 | to 1 ml | | | |

The 5 mg/ml Sodium Propionate in Locke-Ringer Solution had the Following Composition

TABLE 2

| Ingredient | mg/ml | Company | Catalogue N° | Lot N° |
|---|---|---|---|---|
| NaCl | 9 mg | Sigma Aldrich | S5886-500G | SZBE3170 |
| KCl | 0.42 mg | Sigma-Aldrich | P5405-500G | SLBH5524V |
| CaCl₂ 2H₂0 | 0.32 mg | Sigma-Aldrich | C8106-100G | SLBH5904V |
| Dextrose | 2 mg | Sigma-Aldrich | D9434-250G | SLBH3471V |
| NaHCO₃ | 0.2 mg | Sigma-Aldrich | S5761-500G | SLBM8267V |
| Sodium | 5 mg | Sigma-Aldrich | P5436-100G | SLBN3602V |
| propionate | | | | |
| Sterile H₂0 | to 1 ml | | | |

### The Test Item was Sodium Propionate.

Identification: Sodium propionate (Sigma-Aldrich, Cat. P5436-100G)
Test Item Name for Report: Sodium propionate
Description: Hygroscopic white powder
Batch Number: Lot. SLBN3602V
Purity (by Perchlorid Acid Titration): 100%
Stability of Test Item in Solution: ≥6 months in Locke-Ringer Solution
Expiry Date (Retest Date): May 2018
Storage Conditions: Room temperature
Safety Precautions: Routine hygienic procedures (gloves, goggles, face mask).

### Test Item Formulation

Identification: Sodium propionate in Locke-Ringer solution
Test Item Name for Report: Proponent Nasal Spray
Description: Clear aqueous colourless liquid
Batch Number: Not applicable—freshly prepared for the study
Purity (HPLC): 100%
Stability of Test Item in Solution: ≥24 months—freshly prepared for the study
Expiry Date (Retest Date): Freshly prepared for the study—no re-use
Storage Conditions: In the refrigerator+4° C.
Safety Precautions: Routine hygienic procedures (gloves, goggles, face mask).

As delivery device the 3K®-System from the company Ursatec Verpackung GmbH (St. Wendel, Germany) was utilized.

### Filling of Delivery Device:

20 ml of freshly sterile filtrated 5 mg/ml Sodium propionate in Locke-Ringer solution (5 mg/ml solution, for short “5 mg/ml”) were added under aseptic conditions into the bottle and leak-proof assembled.

Clinical score of skin lesion was determined blinded by using the protocol outlined below with values given in FIG. 3.

The sum of the four scores given blinded to each subject (n=2) yielded in the clinical score. The results can be seen in FIGS. 1 and 2.

Accordingly, skin health was improved after treatment with Propionate in Locke-Ringer solution following allergic hypersensitivity as determined by a reduced degree of clinical score compared to control treatment.

### EXAMPLE 2—EFFECT OF CARBOXYLIC ACID ON INJURED SKIN

The study aimed at determining whether treatment with propionate promotes skin health of wound infection of injured skin.

Thus, infected wound was treated 12 hours post wound induction with either 140 μl of 50 mM Propionate in Locke-Ringer solution or with 140 μl water control.

Skin health was improved as determined by degree of erythema of injured skin, as can clearly be seen in FIG. 4.

### EXAMPLE 3—LONG-TERM EFFECT OF CARBOXYLIC ACID ON THE SKIN OF INFANTS

The study aims at determining the long-term beneficial effect of a carboxylic acid on the skin of an infant during its life-time. Thus, infants are topically or orally administered a carboxylic acid, preferably immediately after birth. Dosage can vary depending on weight of the new born infant. As a negative control, infants without being administered a carboxylic acid are observed throughout the duration of the study. During the first years of life of the infant, occurrences of diseases transmitted via the skin are recorded and compared to control infants. For example, viral diseases transmitted via the skin or symptoms of contact dermatitis following exposure of allergens are recorded. Thus, all occurrences of eczema, redness of the skin, skin irregularities and the like are recorded and monitored. After a relevant amount of time, for example 1, 2, 3 or 4 years or more, the number of events is compared. The infant administered with a carboxylic acid at a regular, in particular daily, dosage shows a reduced number of events compared to infants not being administered a regular, in particular daily, dosage of a carboxylic acid.

### EXAMPLE 4—CARBOXYLIC ACID IN THE TREATMENT OF THE SKIN

The study aims at determining whether treatment with propionate promotes barrier function of the skin in infants, in particular when the skin is exposed to allergic substances (particularly allergic contact dermatitis).

Allergic hypersensitivity can be induced through contact sensitization with Nickel allergen over a 12 hour time period. 60 hours following last contact with allergen, treatment of inflamed skin of the infant occurs over 8 hours with either 4×140 μl of 50 mM Propionate in Locke-Ringer solution or with 4×140 μl Locke-Ringer control solution. Clinical score of skin lesion can be determined by using protocols known in the art.

### EXAMPLE 5—EFFECT OF CARBOXYLIC ACID ON INJURED SKIN

The study aims at determining whether treatment with propionate promotes barrier function of the skin subsequent to injury.

Thus, infected wound can be treated 12 hours post wound induction with either 140 μl of 50 mM Propionate in Locke-Ringer solution or with 140 μl water control.

1-8. (canceled)

9. A method for treating and/or preventing a skin disease of a patient and/or for alleviating the symptoms thereof, the method comprising administering to a patient in need thereof an effective amount of a pharmaceutical composition comprising a carboxylic acid or a pharmaceutically acceptable salt thereof.

10-15. (canceled)

16. The method of claim 9, wherein the carboxylic acid or a pharmaceutically acceptable salt thereof comprises between two and four carbon atoms.

17. The method of claim 9, wherein the carboxylic acid or pharmaceutically acceptable salt thereof is one or more of acetic acid, propionic acid, butyric acid, isobutyric acid, 2-hydroxyproirinic acid, dilactic acid, 2-benzyloxypropionic acid, 2-(p-nitrophenyl)-oxy-propionic acid, 3-hydroxypropionic acid, 2,3-dihydroxypropionic acid, methyl 3-hydroxypropionate, ethyl 3-hydroxypropionate, propyl 3-hydroxypropionate, benzyl 3-hydroxypropionate, para-nitrophenyl 3-hydroxypropionate, p-nitrobenzyl 3-hydroxypropionate, polyethylene glycol 3-hydroxypropionate, methyl propionate, ethyl propionate, propyl propionate, benzyl propionate, p-nitrophenyl propionate, p-nitrobenzyl propionate, 2-(4-Isobutylphenyl) propionic acid, or a pharmaceutically acceptable salt of any thereof.

18. The method of claim 9, wherein the carboxylic acid or pharmaceutically acceptable salt thereof is one or more of acetic acid, propionic acid, butyric acid, or a pharmaceutically acceptable salt of any thereof.

19. The method of claim 9, wherein the carboxylic acid or pharmaceutically acceptable salt thereof is propionic acid or a pharmaceutically acceptable salt thereof.

20. The method of claim 9, wherein the pharmaceutical composition is administered to the patient topically, intranasally, or orally.

21. The method of claim 9, wherein the pharmaceutical composition is in liquid form or solid form.

22. The method of claim 9, wherein the pharmaceutical composition is in the form of a spray, a gel, a cream, or an aqueous solution.

23. The method of claim 9, wherein the skin disease is dermatitis or eczema.

24. The method of claim 9, wherein the skin disease is an atopic dermatitis or a contact dermatitis.

25. The method of claim 24, wherein the contact dermatitis is an allergic contact dermatitis.

26. The method of claim 25, wherein the allergic contact dermatitis is metal allergy contact dermatitis.

27. The method of claim 26, wherein the metal allergy contact dermatitis is chromium allergy contact dermatitis or nickel allergy contact dermatitis.

28. The method of claim 9, wherein the carboxylic acid or a pharmaceutically acceptable salt thereof is the only active ingredient in the pharmaceutical composition.

29. A method of (i) reducing one or more of skin redness, skin lesions, wrinkles, skin impurities, or skin irregularities in a subject in need thereof or (ii) improvising one or both of skin softness or skin appearance in a subject in need thereof, the method comprising administering to the subject an effective amount of a topical cosmetic composition comprising an effective amount of a carboxylic acid or a pharmaceutically acceptable salt thereof.

30. The method of claim 29, wherein the cosmetic composition is in liquid form.

31. The method of claim 29, wherein the cosmetic composition is in the form of a spray, a gel, a cream, or an aqueous solution.

32. The method of claim 29, wherein the carboxylic acid or a cosmetically acceptable salt thereof comprises between two and four carbon atoms.

33. The method of claim 29, wherein the carboxylic acid or pharmaceutically acceptable salt thereof is one or more of acetic acid, propionic acid, butyric acid, isobutyric acid, 2-hydroxyproirinic acid, dilactic acid, 2-benzyloxypropionic acid, 2-(p-nitrophenyl)-oxy-propionic acid, 3-hydroxypropionic acid, 2,3-dihydroxypropionic acid, methyl 3-hydroxypropionate, ethyl 3-hydroxypropionate, propyl 3-hydroxypropionate, benzyl 3-hydroxypropionate, para-nitrophenyl 3-hydroxypropionate, p-nitrobenzyl 3-hydroxypropionate, polyethylene glycol 3-hydroxypropionate, methyl propionate, ethyl propionate, propyl propionate, benzyl propionate, p-nitrophenyl propionate, p-nitrobenzyl propionate, 2-(4-Isobutylphenyl) propionic acid, or a pharmaceutically acceptable salt of any thereof.

34. The method of claim 29, wherein the carboxylic acid or pharmaceutically acceptable salt thereof is one or more of acetic acid, propionic acid, butyric acid, or a cosmetically acceptable salt of any thereof.

35. The method of claim 29, wherein the carboxylic acid or pharmaceutically acceptable salt thereof is propionic acid or a cosmetically acceptable salt thereof.
